(19) United States
(12) Patent Application Publication
(10) Pub. No.: US 2018/0028058 A1
(43) Pub. Date: Feb. 1, 2018

(54) DEVICE FOR SUPPORTING AN IMAGE-CAPTURING APPARATUS FOR MEDICAL EXAMINATION AND METHOD

(71) Applicants: Gabriela Roxana SAIDMAN, Berazategui Buenos Aires (AR); Guillermo Andres MONTEOLIVA, La Plata Buenos Aires (AR)

(72) Inventors: Gabriela Roxana SAIDMAN, Berazategui Buenos Aires (AR); Guillermo Andres MONTEOLIVA, La Plata Buenos Aires (AR)

(21) Appl. No.: 15/221,097

(22) Filed: Jul. 27, 2016

(51) Int. Cl. A61B 3/14; A61B 3/00; A61B 5/00

(52) U.S. Cl. CPC A61B 3/14; A61B 5/6898; A61B 3/0041; A61B 5/0077; A61B 5/0022; A61B 5/002

(57) ABSTRACT

A device for supporting an image-capturing apparatus for recording ophthalmologic images and method for recording ophthalmologic images, which allows obtaining and recording images of the retina without need for auxiliary personnel, allowing in turn the live transmission of ophthalmological examination by the provision of an image-capturing apparatus arranged between the eyes of professional and the patient.

### PRIOR ART OF THE INVENTION

### Field of the Invention

The present invention relates to the field of devices, apparatuses and arrangements employed in monitoring, recording and viewing/observing manual tasks in any area such industry, house, medicine and the like, where counting with the hands free to use in the task is necessary, and more particularly relates to a device that allows to carry out visual examinations on a part of a patient under study, and even more particularly refers to a device and method for ophthalmological examination where the use of the two hands of the examining physician is necessary, and wherein a wide-angle camera or cell phone, more precisely a smart phone popularly known as “Smartphones”, can be mounted, which are able to take images, process them and send them at will, or to transmit live to other locations or devices the images that are recorded during the ophthalmological examination.

While most of the following description of the invention is related to medical examinations carried out by the hands of a medical professional, the inventive device is not restricted to this field but it is also suitable for employing in any manual task wherein the worker/examiner have to observe, record and/or transmit the images of the tasks carried out by his/her hands.

### Description of the Prior Art

Before commencing with the description of the devices and systems known in the art and used to help in ophthalmological examinations, it would be appropriate to make references to technical concepts that will help to better understand the purpose and content of the invention.

The study of visual diseases requires, at least in the early stages, the visual examination of eyes by devices that allow the healthcare provider to see them in detail, for example, by using lenses or magnifying glasses which magnify the eye sphere. These magnifying glasses are very well known in the art and are used in combination with other lighting devices, capable of emitting light beams which affect the eyeball and return through the lens, enlarged images that allow the professional to assess the case.

This visual examination is made, among known techniques, through indirect ophthalmoscopy which employs the so-called Indirect Ophthalmoscope which is a device for medical application that uses a light source directed into the eye of the patient by an adjustable mirror, whereby light reflected is joined by a condensing lens to form a real inverted image of the retina. It provides high intensity illumination, which allows to perform a fundoscopy even through turbid optical media, stereoscopic vision and a wide visual field, as well as a dynamic assessment of vitreoretinal pathology. In short, indirect binocular ophthalmoscope uses prisms and coaxial light that allow to see through a magnifying glass the retina, optic nerve, and vitreous in binocular form to evaluate all kinds of diseases of the fundus. Because of its strong light it also allows to evaluate patients with cataract or opaque media.

Moreover, the wide spectrum of use of cell phones is well known, more precisely those smart phones that are used for capturing images in various areas of industry, home, and medicine. An attempt to use these phones in the area of eye examinations was made, but the problem connected with the need of both hands to manipulate the patient's head, eyelids, etc, had to be faced.

By virtue of the current state of the art and its problems, it would be highly desirable to have a new technology that allows the use of smart phones for recording images in eye examinations and more particularly to record retinal images being able to use both hands to manipulate the patient without need for the help of any assistant, also allowing to process and have those images in telecommunication networks in real time or for registration databases.

### BRIEF DESCRIPTION OF THE INVENTION

It is therefore an object of the present invention to provide a new device for viewing, recording and transmitting images of a manual task, such as ophthalmologic images during a medical examination, and more particularly of the retina of a patient, using a smart cell phone or a wide-angle camera, which allows for the use of both hands of the practitioner to auscultate the patient.

It is yet another object of the present invention to provide a device which avoids the use of auxiliary personnel to support the image-capturing apparatus.

It is still another object of the present invention to provide a device that allows the live transmission or “streaming” of ophthalmological examination.

It is a further object of the present invention to provide a device provided with a flexible arm that allows to regulate the distance between the image-capturing apparatus and the patient's eyes.

It is still a further object of this invention to provide a device for the live transmission of ophthalmological examination, allowing telemedicine sessions in remote places of the country, in severe cases.

It is a further object of the present invention to provide a device that may achieve wide-angle retinal images economically, efficiently and safely, using technology available to any individual.

It is still another object of the present invention to provide a device for being used in telemedicine and extendable to all infants and children under 3 years with other diseases.

It is a further object of this invention to provide a device for supporting an image-capturing apparatus for recording ophthalmologic images, which comprises at least a helmet or holder adaptable to the head of a person which is provided with at least an arm extending from and beyond the helmet, said arm being connected to at least one support for at least a image-capturing apparatus.

It is a further object of this invention to provide a device for supporting an image-capturing apparatus for medical examination and recording images of at least part of the body of a patient under examination, the device comprising at least a helmet or head-mounted holder adapted to be affixed to the head of an examiner with the helmet being provided with at least an arm extending from and beyond the helmet, said arm having a distal end and at least one support for at least an image-capturing apparatus is connected to the distal end of the arm.

It is another object of this invention to provide a device for supporting an image-capturing apparatus for viewing and recording/transmitting images of a task that is being carried out by a person by using his/her hands, the device comprising at least a helmet adapted to be affixed to the head of the person carrying out the task with the helmet being provided with at least an arm extending from and beyond the helmet, said arm having a distal end and at least one support for at least an image-capturing apparatus is connected to the distal end of the arm.

It is another object of the invention to provide a method for recording images during medical examinations by means of a device for supporting an image-capturing apparatus for medical examination and recording images of at least part of the body of a patient under examination, wherein the device comprises at least a helmet adapted to be affixed to the head of an examiner with the helmet being provided with at least an arm extending from and beyond the helmet, said arm having a distal end and at least one support for at least an image-capturing apparatus is connected to the distal end of the arm, the method comprising the steps of:

placing the helmet on the head of the examiner,

adjusting the helmet until a comfortable position is obtained,

connecting the image-capturing apparatus to a display screen via a cable,

mounting the image-capturing apparatus on the support,

positioning the arm so that the image-capturing apparatus remains intercalated in line between eyes of the examiner and the at least part of the body of the patient,

examining the patient to find any abnormality,

capturing or recording images of the patient using a mobile application installed on the image-capturing apparatus,

viewing in real time said images on the display screen, and

making a diagnosis.

It is still a further object of this invention to provide a method for recording ophthalmologic images through the use of a device for supporting an image-capturing apparatus for recording ophthalmologic images which comprises at least a helmet adaptable to the head of a person which is provided with at least an arm extending from and beyond the helmet, said arm being connected to at least one support for at least a image-capturing apparatus, and wherein the method comprises the steps of placing the helmet on a head, adjusting the helmet until a comfortable position is obtained, connecting the image-capturing apparatus to a display screen via a cable, mounting the image-capturing apparatus on the support, positioning the arm so that the image-capturing apparatus remains intercalated in line between the eyes of a professional and the eyes of a patient, examining the patient's eye to find any abnormality, capturing or recording images of the eye using a mobile application installed on the image-capturing apparatus, viewing in real time said images on the display screen, and making a diagnosis.

### BRIEF DESCRIPTION OF DRAWINGS

For clarity and understanding of the object of the present invention, it has been illustrated in several figures, in which the invention has been represented in one of the preferred embodiments, all by way of example, wherein:

FIG. 1 shows a side view of the device according to the present invention, wherein an examiner or professional performing ophthalmologic examination to a patient is illustrated;

FIG. 2 shows a top view of the device of the invention, in which a smart phone is illustrated;

FIG. 3 shows a perspective view of the device of FIG. 2 and in accordance with the present invention;

FIG. 4 shows a side view of the device of the present invention wherein a wide-angle camera that is connected to a display screen is illustrated;

FIG. 5 shows a side view of a second alternative embodiment of the device of the present invention wherein an articulated arm is illustrated.

### DETAILED DESCRIPTION OF THE INVENTION

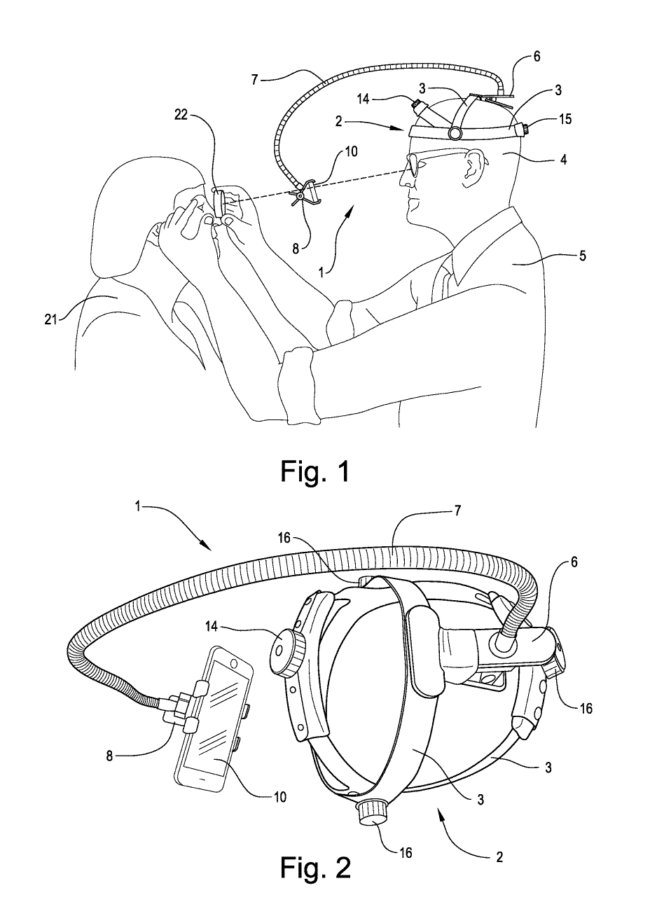

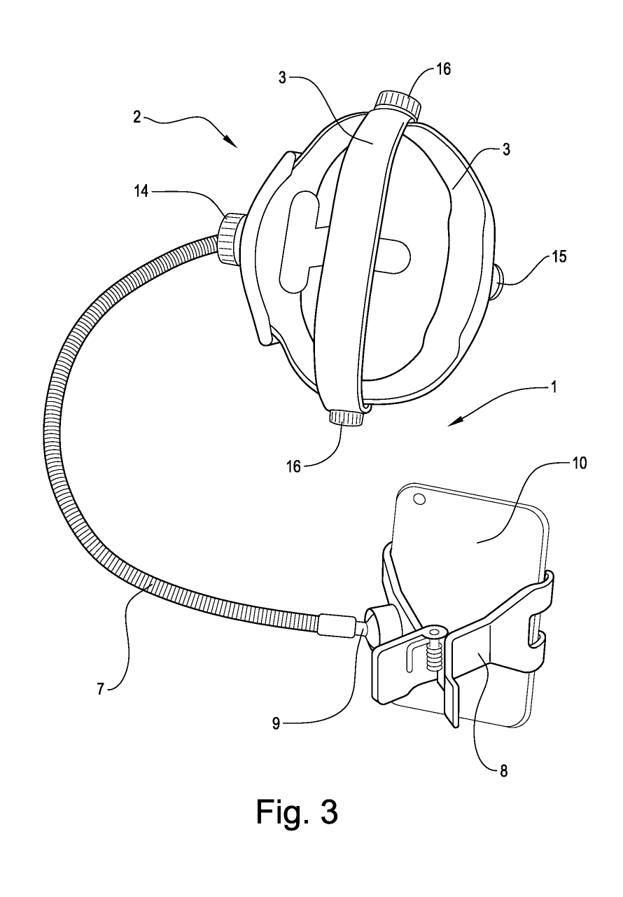

Referring now to FIGS. 1 to 5, it is seen that the invention consists of a new device for supporting a image-capturing apparatus for recording ophthalmologic images, which is indicated by the general reference 1 and comprises at least a helmet 2 defined by adjustable harnesses 3 which adjust and adapt to the head 4 of a professional 5, as best illustrated in FIG. 1. Wherein, said helmet 2 has front 14, rear 15 and side 16 adjustment/regulating means, which remarkably facilitate adjustment of the helmet 2 on the head of the examiner or professional. For the purposes of this invention, helmet is any kind of band, strap or holder capable of being mounted in the head of the examiner, worker, physician, etc.

Likewise, said helmet 2 is provided with a base 6 from which at least one arm 7 extends and projects. The base can be fixedly or detachable mounted, while preferably said arm 7 can be any type of flexible arm used and well known in the photographic industry, which comprises a truncated cylindrical or conical tube, hollow inside, and designed for making it possible to direct light, such as a smart phone, diffusely—if necessary placing filters thereon—and coaxial to avoid shadows on the wide angle image.

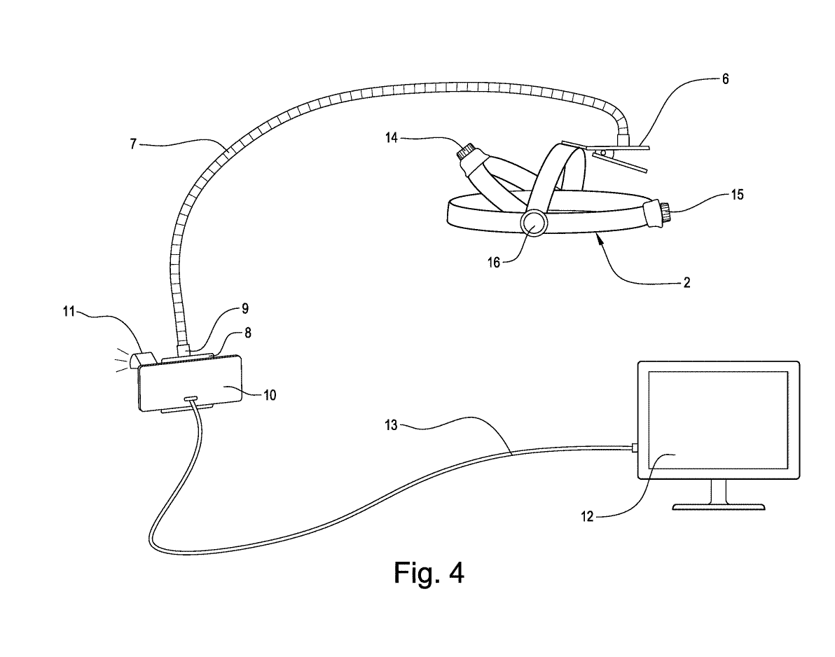

Arm 7 has a distal end at which a support 8 is connected to the flexible arm 7, preferably via an adapter 9, in which an image-capturing apparatus 10 is housed, mounted or removably retained. Said image-capturing apparatus 10 can comprise a smart phone, FIGS. 1 to 3, or either, a wide-angle camera, FIG. 4. In the case of using a wide-angle camera, it is provided with an external source of light 11. Adapter 9 is preferably one that can be moved in all directions to permit moving support 8 to place apparatus 10 in a desired position for viewing and recording.

It is emphasized that the support 8 has a shape similar to housing that protect “Smartphones” or smart phones, with holes for camera and flash LED lamp. It is made of safe and durable material. Support 8 places the cell phone with the camera in perfect axis with the hole and camera and flash and in perfect axis with the flexible arm, so that images and light are not distorted. It is noteworthy that support 8 can be any type of support to help securing the smart phone, not being the invention limited to the one described above.

For its part, adapter 9 comprises a coupling system or thread that can secure the support with the flexible arm. If necessary, to improve image quality, internal light exiting the cell can optionally be focused by filtering system to make it diffuse, or by a system of rotating mirror controlled from the flexible arm, to center it, similar to the system of indirect ophthalmoscopes used in ophthalmology.

Both the wide-angle camera and cell phone can be connected to a display screen 12 by a WiFi connection, Bluetooth or HDMI cable 13. In this way, one can perform the collection or record of images of the retina, as well as, the live transmission or “streaming” of ophthalmological examination to be performed on a patient 14. Live transmission or “streaming” of the examination allows better training of professionals or future professionals, as well as the possibility of live teleconferences in Congresses that are held worldwide.

Alternatively, if the camera is mounted on the helmet, it is located at the base 6. In this way, through an HDMI connection, one projects the image to an external display, similar to a smart phone, but with a wide angle image, convenient to view the retina.

While FIG. 4 illustrates a wide-angle camera 10 connected to a display screen 12 via a HDMI cable 13, this does not imply that the invention is limited to such a configuration, but the smart cell phone can also be connected to the display using the HDMI cable 13 without any problem.

It is worth highlighting that the device of the present invention can operate with a magnifying glass 15 that allows you to view the retina, optic nerve, and vitreous, which is well known and used in the field of art and that for these reasons, we shall not go into descriptive details about the structural configuration thereof. Such magnifying glass can be of 28 diopters or 40 diopters, including 20 diopters. Wherein, it is known that the lower diopter power of the magnifying glass, the greater image and lower field and vice versa.

Also, the support 8 has such a structure that it allows the passage of a beam of light coming from the cell phone itself or external light source so that it may affect the fundus of the patient and allow to capture images using the camera or cell, showing these images on the screen of the smart cell phone.

Thus, the professional puts on the helmet which holds the image-capturing apparatus; adjusts it by means of adjusting means of harnesses in the most comfortable position so that it may not cause fatigue during the examination; then connects the cell phone to the display screen through the respective cable; places the cell phone on the support and simultaneously positions the arm in such a way to intercalate the cell phone in line between the eye of the professional and the patient; and looks at or examines the patient's eye through the cell phone screen or display screen, what he/she seeks with the magnifying glass, either in a video mode or repetitive photos to find an abnormality in it. Consequently, he/she records images that he/she sees on the screen and leaves recorded the video or then performs the capture of photos. Finally, she/he makes a diagnosis. It is emphasized that, the cell phone is turned on and the application of “camera” is previously open to be mounted on the support. As it shall be described below, the cell can also operate with different types of mobile applications to facilitate image registration of the eye. Also, the professional can use the magnifying glass in conjunction with the cell phone if desired, positioning it in an intercalated way between the patient's eye, cell phone and professional's eye.

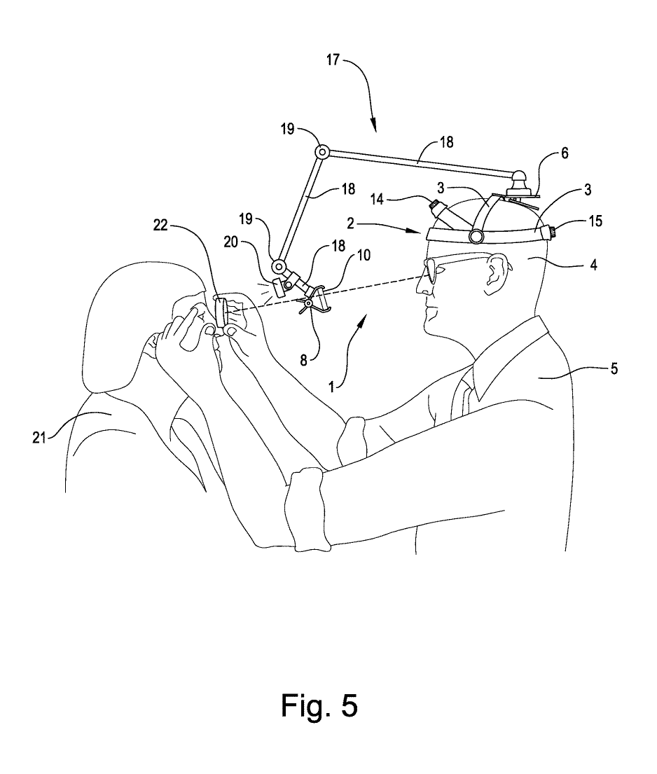

While there is illustrated a flexible arm 7, this does not imply that the invention is limited thereto, but other types of arms can also be considered and used, such as for example, robotic arms, articulated arms or a combination thereof. According to FIG. 5, but not limited to the invention, a second preferred embodiment of the device of the invention can be seen, wherein an articulated arm 17 is illustrated which comprises three sections 18 articulated at pivot points 19. Likewise, said articulated arm 17 may be provided with an external source of light 20 which is mounted on one of the sections 18 of the arm 17 and having a pivotal movement to adjust the light beam in the right direction in order to facilitate the examination of the patient's eye.

Thus, the device of the present invention is constituted and built, wherein thanks to the arrangement of the arm, the image-capturing apparatus 10 can be placed between the eyes of professional and patient to perform the ophthalmologic examination, making it possible that the professional in charge can use both hands without any problem and, more particularly, without the need of having an auxiliary staff to hold the cell phone or camera.

Consequently, the doctor can examine the area to be controlled, looking for and rotating the eyeball by himself/herself, thus avoiding the lack of coordination or understanding between the professional and the assistant/auxiliary staff. Also, having both hands free, the indirect binocular ophthalmoscopy (OBI) can be carried out without any problem, making the learning curve easier. It is so that, soon after the observer uses the device of the invention it is almost impossible to register with the phone only (more difficult learning curve). On the other hand, having one's hands free and able to use the OBI technique, one can access peripheral lesions rotating the patient's head, indicating the gaze direction if he/she collaborates, or if he/she does not collaborate, rotating the eyeball in the clockwise direction of the fundus where the lesion is located.

It is emphasized that, if each work team of a network has a registration method, severe cases can be communicated and consulting can be done. Retinopathy of premature infant has areas with difficulties in research and consultancy and for these reasons, it is advisable to use the device of the invention. In turn, the device of the invention is useful for the prevention of blindness due to retinopathy and severe retinal diseases such as tumors, such as retinoblastoma, so its usefulness has a major positive impact on the health of child growth and development. Furthermore, it also helps to prevent serious forms of eye diseases such as retinopathy of premature infant, ocular tumors, nervous system tumors, assessment in seriously ill inpatients and inpatients with traumatisms.

In an example of application, the invention can be used in cases of ROP injury, peripheral, retinopathy of premature infant, being important for late or regressing ROP wherein evolution can be recorded and compared. Control of a premature baby is performed, wherein thanks to the use of the device of the invention, the professional has both hands free to rotate the eyeball and control the evolution of Retinopathy of Premature infant grade 1 in zone III (peripheral) as it is usually performed around the world with the OBI. There is observed a white line, line of demarcation of the ROP 1 which will lighten in the baby's controls when vessels grow towards periphery. Registration allows to compare images, explain to parents what their child has in real time, improving adherence to monitoring controls and doctor-patient relationship.

In another application example, we can mention the use of the invention for detecting a peripheral lesion of a newborn with Congenital Toxoplasmosis, to whom positive serology for infection was detected and Toxoplasmosis peripheral active focus OBI was detected, wherein treatment commencement was indicated. Recording by device of the invention was performed and control registration was carried out on the week in which treatment was started. It was observed in the equator area of the eyeball an even active lesion and a small focus already pigmented (healed). A both hands of the professional are free, it was possible to use the indenter to rotate, in this case, the eyeball and be able to view and record through the Smartphone screen, as it had already been seen during the diagnostic examination through the indirect ophthalmoscope binocular. In cases where the injury is more peripheral, the globe can be indented so as to be seen.

Contrary to cases above, without using the device of the invention, more hands form assistants are needed to achieve records. This makes it very difficult, if not impossible, to record peripheral lesions of the fundus, where a free hand, ideally from the observer, is required to rotate/indent the eyeball.

By way of example, but not limitative to the present invention, the external light source may be a Pocket Steady LED Camera Manfrotto m1120-1, Camera Light & Smartphone. The flexible arm can be a Manfrotto 237HD Heavy-Duty Flex Arm—for Super Clamp, or Manfrotto 237 (2896) Flex Arm for Super Clamp. The support may be a Manfrotto Klyp Base—Custodia x iPhone 4/4S+LED Light 240 and cavalletto Pocket, or a Manfrotto Klyp Base—Custodia x iPhone 4/4S+LED Light 240 and cavalletto Pocket. The wide-angle camera may be any video camera or those known as “GoPro”.

Although a device for use in ophthalmology has been described, it should be noted that it can be used, through the use and adaptation of accessory, with other wide angle magnifying glasses used in general ophthalmology and for the prevention of other diseases such as diabetes, maculopathy, artery disease, tumors, neuro-ophthalmology, trauma, retinal detachment, various diseases, inherited retinal degenerations, congenital malformations, etc.

Likewise, each of the components comprising the invention can be disassembled and assembled without any inconvenience in order to be able to perform sterilization or hygiene.

In alternative and illustrative manner, the invention can be connected to or use a mobile registration system or application for patient safety and better handling of images. The mobile application allows secure data recording (using bar codes or IQ); Family or patient authorization for the Registry attached to the study (using system for scanning signed authorization, integrated into the APP), record of epidemiological data (which can be compatible with data for sending to epidemiology centers); Image recording and editing thereof; storage on the device and subsequent delivery to a backup system; or data encryption (to secure the identity of images and patient).

By way of example, but without limiting the invention, mobile professional applications (App) for video and edition with smart phone can be used. For example, the mobile application FILMIC Pro, now available for IOS and android systems makes it possible to pre-set high-resolution video parameters, for example 4K, Slow, etc., already known in these phones. But as the device has the helmet and the arm with a predetermined work distance, setting studies can be done for different colors of retina, according to the amount of pigment that it ma have, as well as the presetting of focus, range, white balance, etc., allowing an easier use, better learning curve and better performance (yield) in the registry. Since usually the factory default video mode is used, and often the image delays in being accommodated or there are “burned” images due to much light, etc. (e.g. a injury of retinoblastoma calcified tumor that “burns image” by reflectivity of light of led flash video by calcium, can be set with low white balance gain, etc.).

It is so that the invention allows reviewing studies as often as you want because of doubts about clinical aspects of injuries and parts of the fundus, just reviewing the video. In the running, paused mode, when detecting the lesion one can go back and forth to observe the light reflectivity on ocular tissues. Even, you can detect additional injuries that may be overlooked in an OBI review when one pays more attention to main injury, thereby increasing the importance in diagnosis. In this sense, transmitting to a monitor or Smart TV in real time allows that the case be assessed at the time by several observers in a team with a single observation (higher quantity of observers), making an athenaeum in real time in complex cases. Also, you can add the “streaming” system of Smartphones for use in Telemedicine in e-health networks (consultancies in remote regions for severe cases, with Base Hospitals). The invention is an excellent complement to networks, with informed consent in severe cases, since it allows you to record very peripheral injuries and promoting adherence of the patient and family to attend follow-up checks of investigations or post-treatment controls by using comparative images.

The helmet or headband-mounted holder for a smartphone, according to the invention, gives to the observer the possibility to obtain a steady and neat view of the ocular fundus through a smartphone and different types of condensing lenses (+20 D, +28 D, or a +40 D), with different degrees of field of view and magnification. It proves to be very comfortable and safe for digital indirect fundoscopy, giving the examiner the possibility to freely use one of her/his hands for scleral indentation and globe rotation during examination. With these maneuvers, the far periphery of the fundus can be nicely observed and the globe kept steady for better video capturing. Good quality fundus pictures from video clips may be obtained at any moment during the examination. Also, during examination the fundus image may be observed simultaneously on a TV screen.

Although smartphone fundoscopy can be achieved with multiple techniques, the present device for smartphone proved to be a very simple to use, comfortable, and useful device for hands-free digital indirect funduscopy through a smartphone in different clinical situations, and may have a high impact in clinical practice, especially in settings without the availability of sophisticated fundus imaging technology for telemedicine and teaching. Notably, the device of the invention may also be used in conjunction with devices placed in front of the eyes to see 3D images.

We claim:

1. A device for supporting an image-capturing apparatus for medical examination and recording images of at least part of the body of a patient under examination, the device comprising:
at least a helmet adapted to be affixed to the head of an examiner with the helmet being provided with at least an arm extending from and beyond the helmet, said arm having a distal end and at least one support for at least an image-capturing apparatus is connected to the distal end of the arm.

2. A device according to claim 1, wherein said support is connected to said arm via an adapter.

3. A device according to claim 2, wherein said arm comprises an arm selected from the group consisting of flexible arm, robotic arm, articulated arms or a combination thereof.

4. A device according to claim 1, wherein said adaptable helmet comprises two harnesses that surround the head of the examiner and a base on which one end of said flexible arm is mounted.

5. A device according to claim 1, wherein said image-capturing apparatus is a smart cellular phone.

6. A device according to claim 1, wherein said image-capturing apparatus is a wide-angle camera.

7. A device according to claim 6, wherein said wide-angle camera is provided with an external source of light.

8. A device according to claim 1, wherein said image-capturing apparatus is connected to a display screen through a WiFi connection or Bluetooth.

9. A device according to claim 1, wherein said image-capturing apparatus is connected to a display screen through a HDMI cable.

10. Method for recording images during medical examinations by means of a device for supporting an image-capturing apparatus for medical examination and recording images of at least part of the body of a patient under examination, wherein the device comprises at least a helmet adapted to be affixed to the head of an examiner with the helmet being provided with at least an arm extending from and beyond the helmet, said arm having a distal end and at least one support for at least an image-capturing apparatus is connected to the distal end of the arm, the method comprising the steps of:
placing the helmet on the head of the examiner,
adjusting the helmet until a comfortable position is obtained,
connecting the image-capturing apparatus to a display screen via a cable,
mounting the image-capturing apparatus on the support,
positioning the arm so that the image-capturing apparatus remains intercalated in line between eyes of the examiner and the at least part of the body of the patient,
examining the patient to find any abnormality,
capturing or recording images of the patient using a mobile application installed on the image-capturing apparatus,
viewing in real time said images on the display screen, and
making a diagnosis.

11. Method for recording ophthalmologic images according to claim 10, wherein prior to the step of placement of the helmet, the image-capturing apparatus is turned on.

12. Method for recording ophthalmologic images according to claim 11, wherein after turning the image-capturing apparatus on, mobile application opens.

13. Method for recording ophthalmologic images according to claim 10, wherein after the step of positioning the arm, a magnifying glass is positioned on line between the patient and the image-capturing apparatus.

14. Method for recording ophthalmologic images according to claim 10, wherein the step of capturing the image comprises capturing the image and sending it to one or more remote display devices.

15. A device for supporting an image-capturing apparatus for viewing and recording/transmitting images of a task that is being carried out by a person by using his/her hands, the device comprising:
at least a helmet adapted to be affixed to the head of the person carrying out the task with the helmet being provided with at least an arm extending from and beyond the helmet, said arm having a distal end and at least one support for at least an image-capturing apparatus is connected to the distal end of the arm.
